(19) United States
(12) Patent Application Publication
(10) Pub. No.: US 2024/0216069 A1
(43) Pub. Date: Jul. 4, 2024

(54) SYSTEMS AND METHODS FOR CALCULATING TISSUE RESISTANCE AND DETERMINING OPTIMAL NEEDLE INSERTION PATH

(71) Applicant: Covidien LP, Mansfield, MA (US)

(72) Inventor: Premkumar Rathinasabapathy Jagamoorthy, Hyderabad (IN)

(21) Appl. No.: 18/558,392

(22) PCT Filed: May 17, 2022
(86) PCT No.: PCT/US2022/029552

§ 371 Date: Nov. 1, 2023

Related U.S. Application Data
(60) Provisional application No. 63/189,798, filed on May 18, 2021.

(51) Int. Cl. A61B 34/10; G16H 20/00; G16H 30/40

(52) U.S. Cl. CPC A61B 34/10; G16H 20/00; G16H 30/40; A61B 2034/104; A61B 2034/105; A61B 2034/107

(57) ABSTRACT

A method, and a system performing a method, for determining an optimal needle path includes generating a plurality of candidate needle paths from image data of a patient, extracting a cuboid of image data around each candidate needle path, calculating a tissue resistance index from each cuboid of image data around each candidate needle path, and calculating a value for each candidate needle path based on the calculated tissue resistance index. The candidate needle path with the lowest value is selected as the optimal needle path. The tissue resistance value may be displayed on a display.

### CROSS-REFERENCE TO RELATED APPLICATIONS

This application claims the benefit of the filing date of provisional U.S. Patent Application No. 63/189,798 filed on May 18, 2021.

### FIELD

This disclosure relates to needle insertion pathway planning, and in particular, to systems and methods for calculating a tissue resistance index, for example, to determine an optimal needle path.

### BACKGROUND

Computed tomography (CT) images are commonly used to identify objects, such as physiological structures, in a patient's body. In particular, CT images can be used by physicians to identify malignant tissue or problematic structures in a patient's body and to determine their location within the body. Once the location is determined, a treatment plan can be created to address the problem, such as planning a pathway into the patient's body to remove malignant tissue or planning procedures for accessing and altering the problematic structures. Ablation of tumors is an example of a more targeted approach to tumor treatment. In comparison to traditional body-wide types of cancer treatment, such as chemotherapy, ablation technologies are more targeted and limited, but are just as effective. Thus, such approaches are beneficial in providing targeted treatment that limits unnecessary injury to non-problematic tissue or structures in the patient's body, but they require the assistance of more complex technical tools. Accordingly, there continues to be interest in developing further technical tools to assist with targeted treatment of tissue or structural problems in a patient's body.

### SUMMARY

This disclosure relates generally to needle insertion pathway planning, and in particular, to systems and methods for calculating a tissue resistance index, for example, to determine an optimal needle path.

In an aspect, a method for determining an optimal needle path includes generating a plurality of candidate needle paths from image data of a patient, extracting a cuboid of image data around each candidate needle path, calculating a tissue resistance index from each cuboid of image data around each candidate needle path, and calculating a value for each candidate needle path based on the calculated tissue resistance index. In an aspect, the candidate needle path with the lowest value is selected as the optimal needle path.

In an aspect, the method includes displaying the candidate needle paths relative to the image data of the patient on a display.

In an aspect, the method includes displaying the optimal needle path relative to the image data of the patient on a display.

In an aspect, the value is displayed as at least one of a number or color corresponding to the value on a display.

In an aspect, calculating the tissue resistance index from each cuboid of image data around each candidate needle path includes applying average filters to remove noise from each cuboid of image data around each candidate needle path.

In an aspect, calculating the tissue resistance index from each cuboid of image data around each candidate needle path includes measuring homogeneity of the cuboid of image data around each candidate needle path to calculate a standard deviation. Additionally, or alternatively, measuring the homogeneity of the cuboid of image data around each candidate needle path includes generating grey level co-occurrence matrix textures for each 3D image slice of each cuboid and performing a connected component analysis of the cuboid to calculate a number of labeled components. In an aspect, the tissue resistance index is 1 if the number of labeled components from the connected component analysis is more than 3 and if the standard deviation is greater than 0.5, and the tissue resistance index is 0 if the number of labeled components from the connected components analysis is less than 3 and if the standard deviation is less than 0.5.

In an aspect, generating grey level co-occurrence matrix textures for each 3D image slice of each cuboid includes calculating entropy data for each 3D image slice of each cuboid. The entropy data may be used as an input for a connected component analysis.

In another aspect of the disclosure, an ablation system includes a computing device including a processor and a display operably coupled to the computing device and configured to display at least one user interface. The processor of the computing device is configured to extract a cuboid of image data of a patient around each candidate needle path of a plurality of candidate needle paths, calculate a tissue resistance index from each cuboid of image data around each candidate needle path, and calculate a value for each candidate needle path based on the calculated tissue resistance index. The display is configured to display each candidate needle path and the calculated value for each candidate needle path relative to image data of the patient.

In an aspect, the value is displayed as at least one of a number or color corresponding to the value on the display.

In an aspect, the computing device is configured to calculate the tissue resistance index from each cuboid of image data around each candidate needle path by applying average filters to remove noise from each cuboid of image data around each candidate needle path.

In an aspect, the computing device is configured to calculate the tissue resistance index from each cuboid of image data around each candidate needle path by measuring homogeneity of the cuboid of image data around each candidate needle path to calculate a standard deviation. Additionally, or alternatively, the computing device is configured to measure the homogeneity of the cuboid of image data around each candidate needle path by generating grey level co-occurrence matrix textures for each 3D image slice of each cuboid and performing a connected component analysis of the cuboid to calculate a number of labeled components. In an aspect, the tissue resistance index is 1 if the number of labeled components from the connected component analysis is more than 3 and if the standard deviation is greater than 0.5, and the tissue resistance index is 0 if the number of labeled components from the connected components analysis is less than 3 and if the standard deviation is less than 0.5.

In an aspect, the computing device is configured to generate grey level co-occurrence matrix textures for each 3D image slice of each cuboid by calculating entropy data for each 3D image slice of each cuboid. The entropy data may be used as an input for the connected component analysis.

In another aspect of the disclosure, a non-transitory computer readable storage medium is provided, storing instructions, which when executed by a processor, cause the processor to extract a cuboid of image data of a patient around a candidate needle path, calculate a tissue resistance index from each image slice of the cuboid of image data around the candidate needle path, and calculate a value for the candidate needle path based on the calculated tissue resistance index.

In an aspect, the processor calculates the tissue resistance index by measuring homogeneity of the cuboid of image data around the candidate needle path to calculate a standard deviation.

In an aspect, the processor calculates the tissue resistance index by generating grey level co-occurrence matrix textures for each image slice of the cuboid and performing a connected component analysis of the cuboid to calculate a number of labeled components.

### BRIEF DESCRIPTION OF THE DRAWINGS

Various aspects and features of this disclosure are described below with references to the drawings, of which:

FIG. 1 is a block diagram of an example ablation system in accordance with aspects of the disclosure;

FIG. 2 is a flow chart illustrating a method for determining an optimal needle path based on a calculated tissue resistance index, in accordance with aspects of the disclosure;

FIG. 3 is a flow chart illustrating a method for calculating a tissue resistance index for the method of FIG. 2;

FIG. 4 is an example user interface displaying a plurality of candidate needle paths in accordance with an aspect of the disclosure;

FIG. 5 illustrates slice images of an example cuboid of image data of one of the candidate needle paths of FIG. 4;

FIG. 6 is an example entropy chart derived from performing an entropy function on one of the slice images of FIG. 5;

FIG. 7 is an example user interface displayable on a display of the system of FIG. 1; and

FIG. 8 is another example user interface displayable on a display of the system of FIG. 1.

### DETAILED DESCRIPTION

Embodiments of this disclosure are now described in detail with reference to the drawings in which like reference numerals designate identical or corresponding elements in each of the several views. As used herein, the term “clinician” refers to a doctor, a nurse, or any other care provider or user of the system and may include support personnel. Throughout this description, the phrase “in embodiments” and variations on this phrase such as “in aspects” generally is understood to mean that the particular feature, structure, system, or method being described includes at least one iteration of the disclosed technology. Such phrases should not be read or interpreted to mean that the particular feature, structure, system, or method described is either the best or the only way in which the embodiment can be implemented. Rather, such phrases should be read to mean an example of a way in which the described technology could be implemented, but need not be the only way to do so.

Proper percutaneous placement of needles into lung, liver and kidney tumors is important for effective radiofrequency ablation, cryoablation and microwave ablation. These are needle-based therapies that depend on the skill level of an interventional radiologists and the success of the procedure depends on the accurate needle insertion and final placement to ensure that the patient's anatomy is not damaged during insertion and that the ablation zone properly encompasses the tumor. Ablation planning software applications typically utilize preoperative scans as an input and still require a clinician's manual analysis of anatomy around the tumor region to determine a suitable needle insertion path. Such an analysis is limited to two-dimensional slice images of 3D image data, and as such, the clinician's decision-making is limited to the anatomy viewable in the two-dimensional slice image displayed. Even if multiple perspectives of two-dimensional slice images are displayed, the clinician can only analyze a single image at a time. In certain cases, such limited insight results in incorrect needle placement, especially in the case of a deep lesion adjacent to critical structures, which not only impacts treatment outcome but also increases radiation exposure of vital structures. Currently, no imaging solution exists to determine optimal needle path in a simple and accurate manner by leveraging information available in preoperative CT DICOM scans.

A CT scan combines a series of X-ray images taken from different angles around a patient's body and uses computer processing to create two-dimensional cross-sectional images (slices) of the bones, blood vessels and soft tissues inside the patient's body. CT scan images provide more detailed information than plain X-rays. Pixels in an image obtained by CT scanning are displayed in terms of relative radiodensity. The pixel itself is displayed according to the mean attenuation of the tissue(s) that it corresponds to, on a scale from +3,071 (most attenuating) to −1,024 (least attenuating), on the Hounsfield scale. A pixel is a two-dimensional unit based on the matrix size and the field of view. When the CT slice thickness is also factored in, the unit is known as a voxel, which is a three-dimensional unit. In accordance with aspects of the disclosure, the dynamic range of detailed information of the 3D volume of the CT scan, is processed to calculate a tissue resistance index for a given needle insertion path. Additionally, when multiple candidate needle paths are available, the calculated tissue resistance index value for each candidate needle path is utilized to determine an optimal ablation antenna or needle insertion pathway.

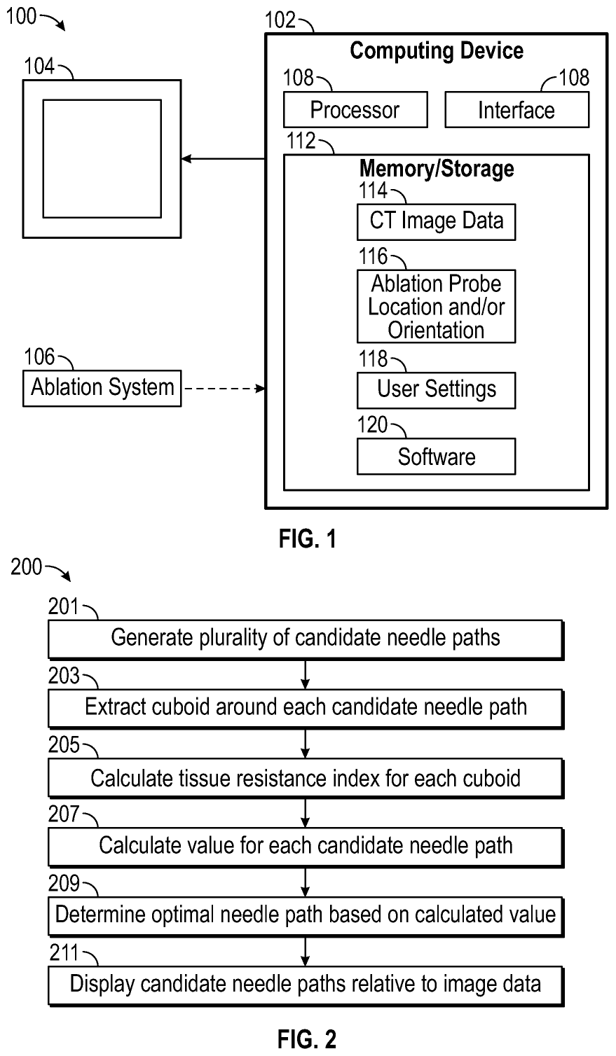

FIG. 1 illustrates a block diagram of a system 100, which includes a computing device 102 such as, for example, a laptop, desktop, workstation, tablet, or other similar device, a display 104, and an ablation system 106. The computing device 102 includes one or more processors 108, interface devices 110 (such as communications interface and user interface), memory and storage 112, and/or other components generally present in a computing device. The display 104 may be touch sensitive, which enables the display to serve as both an input and output device. In various embodiments, a keyboard (not shown), mouse (not shown), or other data input devices may be employed.

Memory/storage 112 may be any non-transitory, volatile or non-volatile, removable or non-removable media for storage of information such as computer-readable instructions, data structures, program modules or other data. In various embodiments, the memory 112 may include one or more solid-state storage devices such as flash memory chips or mass storage devices. In various embodiments, the memory/storage 112 can be RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information and which can be accessed by the computing device 102.

Computing device 102 may also include an interface device 110 connected to a network or the Internet via a wired or wireless connection for the transmission and reception of data. For example, computing device 102 may receive computed tomographic (CT) image data 214 of a patient from a server, for example, a hospital server, Internet server, or other similar servers, for use during surgical ablation planning. Patient CT image data 114 may also be provided to computing device 202 via a removable memory.

In the illustrated embodiment, the memory/storage 112 includes CT image data 114 for one or more patients, information regarding the location and orientation of an ablation probe 116, various user settings 118 (which are described below), and various software that perform the operations described herein 120.

In accordance with an aspect of this disclosure, the software 120 of FIG. 1 includes a treatment planning module which guides a clinician in identifying a target for ablation treatment, a target size, a treatment zone, and a needle path to the target. The treatment planning module can generate a user interface screen for presenting information and receiving clinician input. The clinician can select a patient data set corresponding to a patient via the user interface. The software 120, or another component of system 100, includes instructions which are executable by a processor or any components of system 100 to perform the steps of any of the methods described herein (e.g., method 200 and method 300 described below).

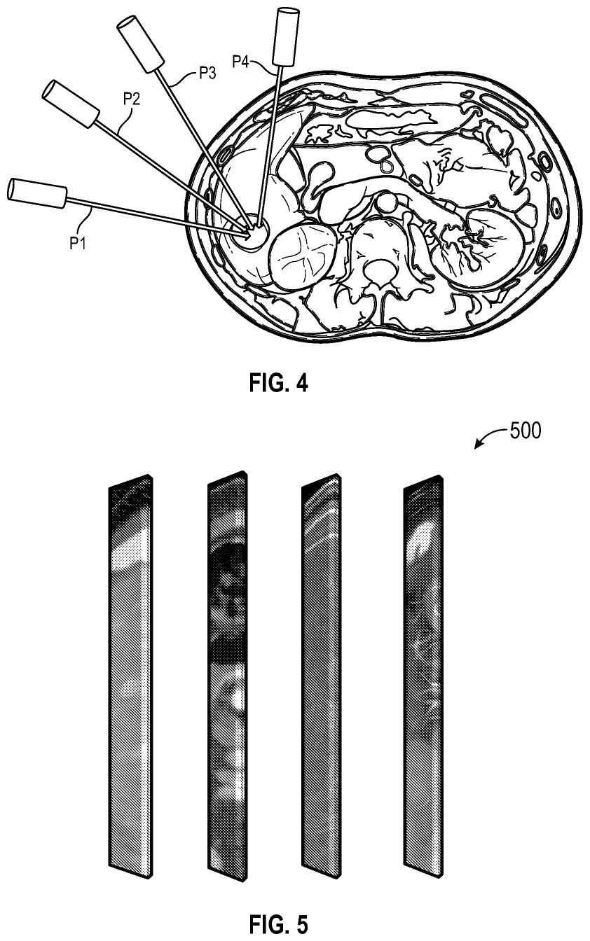

FIG. 2 illustrates a flow chart of a method for determining an optimal needle path based on a calculated tissue resistance index, shown as method 200. Method 200 begins in step 201 where a plurality of candidate needle paths are generated from image data of a patient. The candidate needle paths may be generated manually by a clinician viewing slice image data of the patient or automatically by the system. Candidate needle paths are illustrated in the example user interface shown in FIG. 4 as candidate needle paths P1, P2, P3, and P4. Although method 200 is described as utilizing a plurality of candidate needle paths, it is appreciated that method 200 may be performed utilizing a single candidate needle path, for example, a single candidate needle path that is manipulatable by a user's input.

In step 203, a cuboid of image data (e.g., cuboid 500 illustrated in FIG. 5) is extracted around each candidate needle path. In an aspect, the system crops and extracts 3D image slices around the candidate needle path, for each candidate needle path, by applying and image crop function, providing the starting and ending points in 3D coordinates from the scan data from (x1, y1, z1) to (x2, y2, z2).

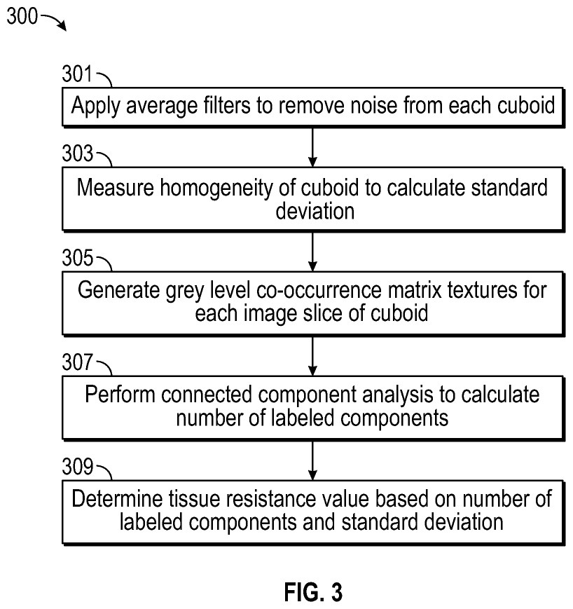

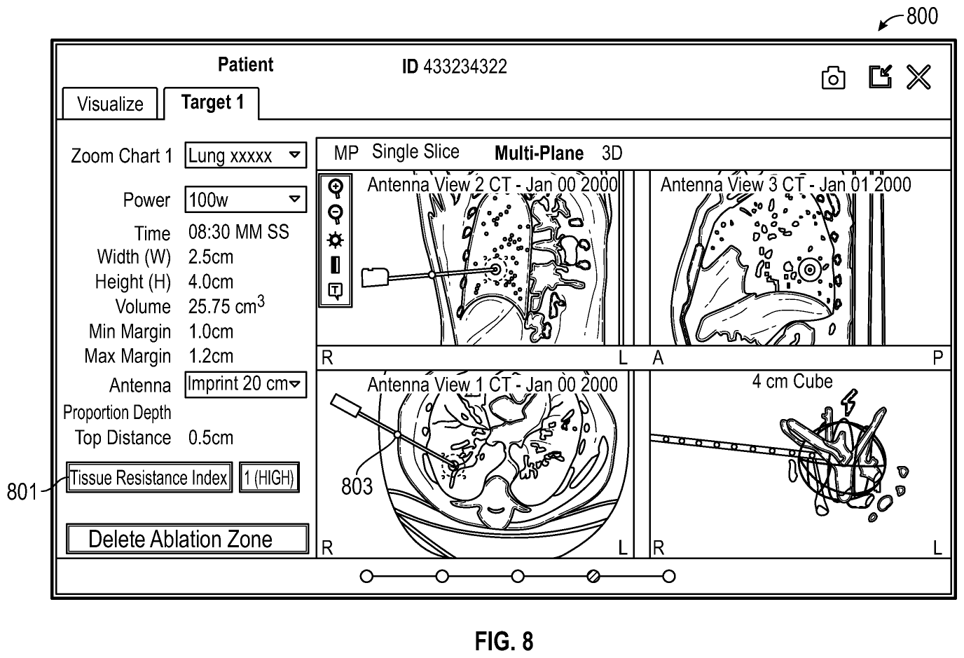

In step 205, a tissue resistance index is calculated for each candidate needle path based on the cuboid corresponding to each candidate needle path. The specific optional sub-steps of calculating the tissue resistance index for each candidate needle path are described in further detail below as method 300 (FIG. 3). In step 207, a value is calculated for each tissue resistance index calculated in step 205. The value of the tissue resistance index calculated in step 207 may be displayed on the display as a numerical value, such as a numerical score, and/or as a visual notification, for example a specific color. An example of a user interface displaying the calculated tissue resistance index value is shown in FIG. 8. In step 209, one needle path of the plurality of candidate needle paths is determined to be the optimal needle path based on the calculated tissue resistance index, for example, the value calculated in step 207. In an aspect, the candidate needle path having a tissue resistance index with the lowest value calculated in step 207 is determined to be the optimal needle path.

In step 211, the candidate needle paths are displayed on a display, for example, overlaid on image data of the patient. An example user interface displayed in step 211 is illustrated in FIG. 4. The candidate needle paths displayed in step 211 may be displayed based on their corresponding tissue resistance value calculated in step 207, for example in a specific color associated with the tissue resistance value. In an aspect, the optimal needle path is displayed in one color, while the remaining candidate needle paths are displayed in a second color to draw the clinician's attention to the optimal needle path.

While the above-described method is described as determining an optimal needle path from a plurality of candidate needle paths, the system may alternatively calculate the tissue resistance index of a single needle path, which may be manipulatable by a user, and display the tissue resistance index value for the single needle path based on its position at a given moment. A user may move the display of the single needle path relative to one or more slice images of the patient and the system may recalculate and display the new calculated tissue resistance value, in real time, as the single needle path is moved by the user. For example, as illustrated in user interface 800 in FIG. 8, the tissue resistance value corresponding to the current position of the needle path 803 is displayed in cell 801. As the user manipulates the needle path 803 (for example, by dragging the needle path along the skin surface in on the axial, coronal, or sagittal CT slice images), the new tissue resistance index value is calculated and displayed in cell 801.

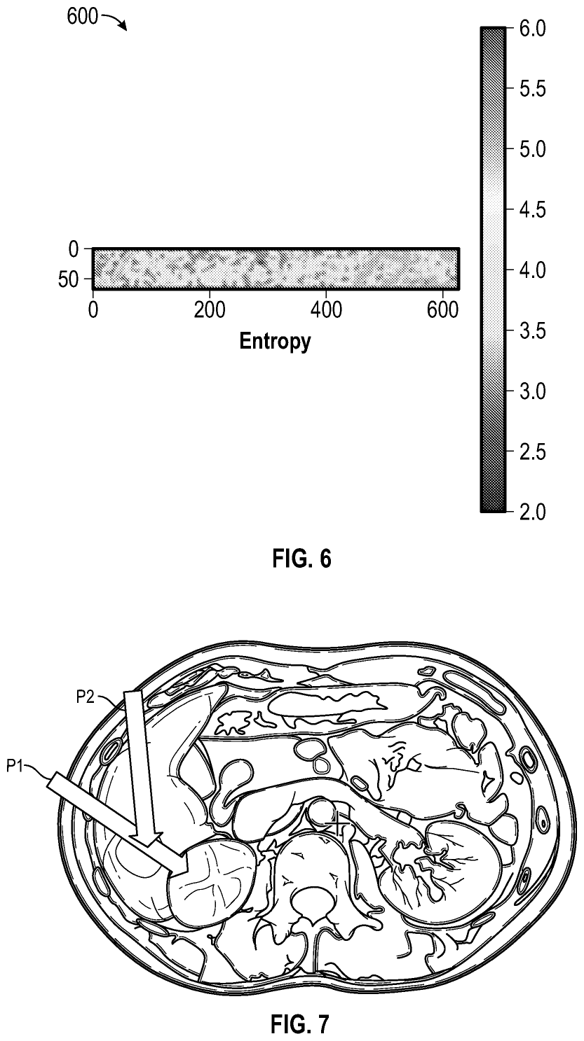

FIG. 3 illustrates a flowchart of one example method for calculating the tissue resistance index (e.g., step 205 of method 200 in FIG. 2), which is described as method 300. Method 300 begins in step 301 where average filters are applied to remove noise from each cuboid of image data around each candidate needle path. In step 303, the homogeneity of the cuboid of image data around each candidate needle path is measured to calculate a standard deviation. In an aspect, the homogeneity of each slice of the cuboid is measured. In step 305, grey level co-occurrence matrix textures for each slice of each cuboid are generated. In an aspect, the system generates grey level co-occurrence matrix textures for 3D image slice and by combining connected component analysis of the preprocessed image segment, calculates the tissue resistance index by leveraging tissue texture characteristics from the CT DICOM scan image. The grey level co-occurrence matrix is used for texture analysis using statistics to measure homogeneity in a region of interest. Here, the region of interest is the needle path image slice of the cuboid for the candidate needle path, which is processed to get the entropy (e.g., one of the statistical parameters computed using grey level co-occurrence matrix). Generating grey level co-occurrence matrix textures for each 3D slice of each cuboid may include calculating entropy data for each 3D image slice of each cuboid, where, for example, the entropy data is used as an input for a connected component analysis described below. FIG. 6 illustrates an example entropy image 600 for one example slice of a cuboid.

In step 307, a connected component analysis is performed to calculate a number of labeled components. In step 309, the tissue resistance value is determined based on the number of labeled components calculated in step 307 and the standard deviation calculated in step 303. In an aspect, the tissue resistance index is 1 (or HIGH) if the number of labeled components from the connected component analysis is more than 3 and if the standard deviation is greater than 0.5, and the tissue resistance index is 0 (or LOW) if the number of labeled components from the connected components analysis is less than 3 and if the standard deviation is less than 0.5. While described as either 0 or 1, it is understood that the value may be calculated between 0 and 1 and not necessarily be binary.

By computing the tissue resistance index for the slice of images along the candidate needle paths (e.g., P1 and P2 in FIG. 4 or a single needle path in FIG. 8), the system computes whether there are variations in image characterization along the pathways. For example, referring to FIG. 7, in candidate needle path P1, as the planned pathway crosses the skin surface, rib bones, liver parenchyma and kidney, the tissue resistance value value is HIGH, whereas for the candidate needle path P2, the tissue resistance index value is LOW as is not crossing through bones and other neighboring organs. Since candidate needle path P1 has the chance of damaging the kidney, while planning for liver ablation, the pathway can be re-adjusted based on the tissue resistance index algorithm to avoid the unwanted damage to the kidney.

The above-described system accelerates the ablation planning process by using a calculated tissue resistance index to quickly identify the most appropriate needle path to reach a target tumor and helps reduce radiation to critical structures. Additionally, the system provides a scale to compare and evaluate different treatment approaches which removes the error associated with manual analysis of anatomy surrounding a target tumor region. The system reduces the possibility of accidental damage to critical regions, for example which may be caused by planning errors, as without the functionality of the system, the user is not able to visualize the needle path from all possible angles while simultaneously accounting for undisplayable data. The disclosed system quickly and quantitatively determines the most appropriate antenna path to reach a target taking into account all data associated with a three-dimensional cuboid, as opposed to data made available by the visualization of a two-dimensional slice image.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope and spirit of the claims appended hereto. For example, while this disclosure refers to some parameters relevant to an ablation procedure, this disclosure contemplates other parameters that may be helpful in planning for or carrying out an ablation procedure including a type of microwave generator, a power-level profile, or a property of the tissue being ablated.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A method for determining an optimal needle path comprising:
generating a plurality of candidate needle paths from image data of a patient;
extracting a cuboid of image data around each candidate needle path;
calculating a tissue resistance index from each cuboid of image data around each candidate needle path;
calculating a value for each candidate needle path based on the calculated tissue resistance index; and
selecting the candidate needle path with the lowest value as the optimal needle path.

2. The method according to claim 1, further comprising displaying the candidate needle paths relative to the image data of the patient on a display.

3. The method according to claim 1, further comprising displaying the optimal needle path relative to the image data of the patient on a display.

4. The method according to claim 1, wherein the value is displayed as at least one of a number or color corresponding to the value on a display.

5. The method according to claim 1, wherein calculating the tissue resistance index from each cuboid of image data around each candidate needle path includes applying average filters to remove noise from each cuboid of image data around each candidate needle path.

6. The method according to claim 1, wherein calculating the tissue resistance index from each cuboid of image data around each candidate needle path includes measuring homogeneity of the cuboid of image data around each candidate needle path to calculate a standard deviation.

7. The method according to claim 6, wherein measuring the homogeneity of the cuboid of image data around each candidate needle path includes generating grey level co-occurrence matrix textures for each 3D image slice of each cuboid and performing a connected component analysis of the cuboid to calculate a number of labeled components.

8. The method according to claim 7, wherein:
the tissue resistance index is 1 if the number of labeled components from the connected component analysis is more than 3 and if the standard deviation is greater than 0.5; and
the tissue resistance index is 0 if the number of labeled components from the connected components analysis is less than 3 and if the standard deviation is less than 0.5.

9. The method according to claim 7, wherein generating grey level co-occurrence matrix textures for each 3D image slice of each cuboid includes calculating entropy data for each 3D image slice of each cuboid.

10. The method according to claim 9, wherein the entropy data is used as an input for the connected component analysis.

11. An ablation system comprising:
a computing device including a processor configured to:
extract a cuboid of image data of a patient around each candidate needle path of a plurality of candidate needle paths;
calculate a tissue resistance index from each cuboid of image data around each candidate needle path; and
calculate a value for each candidate needle path based on the calculated tissue resistance index; and
a display operably coupled to the computing device and configured to display each candidate needle path and the calculated value for each candidate needle path relative to image data of the patient.

12. The system according to claim 11, wherein the value is displayed as at least one of a number or color corresponding to the value on the display.

13. The system according to claim 11, wherein the computing device is configured to calculate the tissue resistance index from each cuboid of image data around each candidate needle path by applying average filters to remove noise from each cuboid of image data around each candidate needle path.

14. The system according to claim 11, wherein the computing device is configured to calculate the tissue resistance index from each cuboid of image data around each candidate needle path by measuring homogeneity of the cuboid of image data around each candidate needle path to calculate a standard deviation.

15. The system according to claim 14, wherein the computing device is configured to measure the homogeneity of the cuboid of image data around each candidate needle path by generating grey level co-occurrence matrix textures for each 3D image slice of each cuboid and performing a connected component analysis of the cuboid to calculate a number of labeled components.

16. The system according to claim 15, wherein:
the tissue resistance index is 1 if the number of labeled components from the connected component analysis is more than 3 and if the standard deviation is greater than 0.5; and
the tissue resistance index is 0 if the number of labeled components from the connected components analysis is less than 3 and if the standard deviation is less than 0.5.

17. The system according to claim 15, wherein the computing device is configured to generate grey level co-occurrence matrix textures for each 3D image slice of each cuboid by calculating entropy data for each 3D image slice of each cuboid, wherein the entropy data is used as an input for the connected component analysis.

18. A non-transitory computer readable storage medium storing instructions, which when executed by a processor, cause the processor to:
extract a cuboid of image data of a patient around a candidate needle path;
calculate a tissue resistance index from each image slice of the cuboid of image data around the candidate needle path; and
calculate a value for the candidate needle path based on the calculated tissue resistance index.

19. The non-transitory computer readable storage medium according to claim 18, wherein the processor calculates the tissue resistance index by measuring homogeneity of the cuboid of image data around the candidate needle path to calculate a standard deviation.

20. The non-transitory computer readable storage medium according to claim 18, wherein the processor calculates the tissue resistance index by generating grey level co-occurrence matrix textures for each image slice of the cuboid and performing a connected component analysis of the cuboid to calculate a number of labeled components.
